(19) United States
(12) Patent Application Publication
(10) Pub. No.: US 2019/0102519 A1
(43) Pub. Date: Apr. 4, 2019

(54) TARGETING ADAPTER FOR MOBILE SCANNING DEVICE

(71) Applicant: Spectralink Corporation, Boulder, CO (US)

(72) Inventor: Christophe Durand, Superior, CO (US)

(73) Assignee: Spectralink Corporation, Boulder, CO (US)

(21) Appl. No.: 15/722,725

(22) Filed: Oct. 2, 2017

(51) Int. Cl. G06F 19/00; A61B 90/96; G06K 7/14; G06K 7/10

(52) U.S. Cl. CPC G06F 19/322; A61B 90/96; G06K 7/10722; G06K 7/1417; G06K 7/1413

(57) ABSTRACT

A scanning apparatus includes a computing device that includes a housing, a camera, and a light source, the computing device being operable to activate the light source and capture optical data using the camera. A scanning adapter is coupled to the computing device and includes a body defining an aperture aligned with the light source and a targeting assembly movably coupled to the body. The targeting assembly includes an optical assembly, such that in a first position of the targeting assembly the optical assembly is misaligned with the aperture and in a second position of the targeting assembly the optical assembly is aligned with the aperture and the light source such that the optical assembly projects a targeting aid at a predetermined distance from the camera in response to activation of the light source.

### TECHNICAL FIELD

Aspects of the present disclosure relate to data capture devices and, in particular, to a targeting adapter for use with a mobile data capture device that provides visual feedback regarding positioning of the mobile data capture device relative to the data being captured.

### BACKGROUND

Modern mobile devices are sophisticated computing devices capable of performing a wide range of functions. In addition to traditional telephonic communication, mobile devices are also capable of collecting and processing visual data for use in various applications. For example, a mobile device may be used to scan or otherwise capture barcodes, QR codes, and similar optical, machine-readable representations of data to facilitate data input and/or tracking of patient information in hospitals, products in warehouses, components in manufacturing facilitates, and the like.

The effectiveness with which a mobile device is able to process visual data is highly dependent on the clarity and accuracy of the captured image data. Out of focus and/or misaligned image data, for example, may increase the time and resources required to process the captured image data and to determine an alphanumeric string or similar data represented by the captured data. In applications involving manual/handheld scanning, such suboptimal image data may result from inconsistency in the positioning and orientation of a scanning device relative to the item being scanned. Placing the scanning device too close or too far from the item, for example, may result in the captured image data being out-of-focus and, as a result, may lead to, among other things, increased processing time and incorrect or incomplete conversion of the image data. To the extent the image data cannot be fully processed, rescanning may be required resulting in wasted time and resources.

With these thoughts in mind among others, aspects of the systems and methods disclosed herein were conceived.

### SUMMARY

According to one aspect of the present disclosure, a scanning apparatus is provided. The scanning apparatus includes a computing device including a housing, a camera, and a light source, the computing operable to activate the light source and capture optical data using the camera. The scanning apparatus further includes a scanning adapter coupled to the mobile computing device and including a body defining an aperture aligned with the light source and a targeting assembly. The targeting assembly is movably coupled to the body between a first position and a second position and includes a lens. In the first position of the targeting assembly, the lens is misaligned with the aperture while in the second position of the targeting assembly the lens is aligned with the aperture and the light source such that the lens projects a targeting aid at a predetermined distance from the camera in response to activation of the light source.

In another aspect of the present disclosure, a scanning adapter is provided. The scanning adapter includes a body defining an aperture and a targeting assembly movably coupled to the body and including an optical assembly. When the targeting assembly is in a first position, the optical assembly is misaligned with the aperture. When the targeting assembly is in a second position, the optical assembly is aligned with the aperture such that the optical assembly projects a targeting aid at a predetermined distance in response to receiving light through the aperture.

In yet another aspect of the present disclosure a method of capturing data using a scanning device is provided, the scanning device including a flash, a camera, and a targeting assembly adapted to project a targeting aid at a predetermined distance when the flash is activated. The method includes activating the flash, receiving a capture command, deactivating the flash in response to receiving the capture command, and, after deactivating the flash, capturing image data using the camera.

### BRIEF DESCRIPTION OF THE DRAWINGS

The foregoing and other objects, features, and advantages of the present disclosure set forth herein should be apparent from the following description of particular embodiments of those inventive concepts, as illustrated in the accompanying drawings. The drawings depict only typical embodiments of the present disclosure and, therefore, are not to be considered limiting in scope.

FIG. 1A is a front plan view of a first scanning device including a scanning adapter having a targeting assembly;

FIGS. 1B-C are rear plan views of the first scanning device illustrating the targeting assembly in a first and second position, respectively;

FIGS. 2A-B are cross-sectional side views of the scanning adapter of FIG. 1;

FIGS. 3A-B are schematic illustrations of the scanning adapter including alternative targeting assemblies;

FIG. 4A is a schematic illustration of a circular target aid that may be projected using scanning devices in accordance with the present disclosure;

FIG. 4B is a schematic illustration of a square target aid including focus symbols that may be projected using scanning devices in accordance with the present disclosure;

FIG. 4C is a schematic illustration of a rectangular target aid including corner alignment features that may be projected using scanning devices in accordance with the present disclosure;

FIG. 4D is a schematic illustration of a linear target aid that may be projected using scanning devices in accordance with the present disclosure;

FIG. 4E is a schematic illustration of a targeting aid including textual features that may be projected using scanning devices in accordance with the present disclosure;

FIG. 5A is a rear plan view of a second scanning device including a second scanning adapter having a targeting assembly in a first position;

FIG. 5B-C are a front and a plan view of the second scanning device, respectively, in which the targeting assembly is in a second position;

FIG. 6 is a cross-sectional side view of the scanning adapter of FIGS. 5A-C;

FIG. 7 is a block diagram illustrating an example scanning device in accordance with the present disclosure; and

FIG. 8 is a flow chart illustrating a method of capturing data using a scanning device in accordance with the present disclosure.

### DETAILED DESCRIPTION

Aspects of the present disclosure are directed to a targeting aid for use with a mobile scanning device and methods of capturing data using the targeting aid. The mobile scanning device may, for example, be a conventional smartphone or similar mobile device including a camera and flash or other light source and on which scanning software or a scanning application may be executed. The targeting aid is coupled to the mobile scanning device and uses light from the flash to project a reticle or similar targeting aid to guide a user in positioning the mobile scanning device relative to the object being scanned for improved capture.

Mobile devices, such as smartphones, often include cameras that may be used to capture and process images including machine-readable optical data representations, such as barcodes. The efficiency and accuracy with which such mobile devices are able to capture and process such data representations is often dependent on the positioning and orientation of the device relative to the object being scanned. For example, if the mobile device is too far or too close to the object being scanned or misaligned with the object, the mobile device may not be able process and interpret the captured image such that rescanning of the object is required.

The efficiency with which barcodes and other data representations may be scanned using a conventional mobile device is further impacted by the need to use a display of the mobile device during scanning to properly align the mobile device with the object being scanned. Requiring a user to “look through” the mobile device to scan an object in this manner limits the useful angles and positions of the mobile device and, as a result, limits the scanning applications for which the mobile device may be used.

In light of the foregoing problem, the present disclosure provides a scanning adapter for use with a mobile scanning device. The scanning adapter includes a targeting assembly that generates a targeting aid to assist a user of the mobile scanning device in positioning the mobile scanning device at an optimal location and in an optimal orientation for performing a scanning task. More specifically, the targeting assembly is movable into a position over a flash or other light source of the mobile device. With the targeting assembly positioned over the flash, the flash may be turned on such that light from the flash passes through an optical assembly of the targeting assembly to project the targeting aid. The optical assembly may include one or more optical elements that focus and shape the light to generate the targeting aid. The optical elements generally include a lens shaped to focus the light at a predetermined distance from the camera corresponding to an optimal scanning distance. The optical elements further include a mask that blocks or otherwise shapes the light to produce a particular shape or light pattern to facilitate alignment of the camera with the object being scanned. The degree of focus of the light pattern further provides feedback to a user regarding the relative placement of the mobile scanning device to the object being scanned.

The scanning adapter may, in certain implementations, be coupled to or otherwise integrated with a mobile device having multiple uses. Such functionality may include, among other things, conducting voice or video calls and taking photographs. Accordingly, to minimize the degree to which the scanning adapter may interfere with such additional functions to the mobile device, the targeting assembly is movable between a position in which the targeting assembly is disposed over or otherwise aligned with the flash or similar light source (i.e., during or in preparation for scanning) and a position in which the targeting assembly is not aligned with the flash or light source. When the targeting assembly is in the latter position, the mobile device may generally be used for other purposes without interference by the targeting assembly.

In certain implementations, the scanning adapter is configured such that the optical assembly is aligned with the flash and scanning is performed with the mobile scanning device held in a substantially vertical position. In other implementations, the optical assembly may be arrange perpendicular to the flash and the targeting assembly may include one or more mirrors to redirect light to and from the mobile scanning device such that scanning may be performed with the mobile scanning device in a substantially horizontal orientation.

By projecting a targeting aid, a user of the scanning adapter disclosed herein is not required to look through the display of the mobile device in order to properly align the mobile device with the object being scanned. Accordingly, the user is able to successfully and efficiently scan objects even if the mobile device is held by the user in a manned that prevents viewing of the display of the mobile device. In a nursing application, for example, a nurse would be able to reach across a patient to scan a wristband or similar identification without having to walk around the patient or otherwise reposition such that the nurse is able to view the wristband through the display of the mobile device.

To capture data using the disclosed scanning adapter, the targeting assembly is first disposed over the flash. The mobile scanning device may then be put into a targeting or similar mode in which the flash is activated such that the light from the flash is used to project the targeting aid. Here, the flash is held on, not briefly flashed, while targeting occurs. Entering into the targeting mode may cause the scanning device to attempt to recognize a barcode or other machine-readable data representation. For example, the mobile scanning device may begin streaming or otherwise transmitting data from a camera to a processor and, more particularly, to a decoding module adapted to identify machine-readable data representations. In certain implementations, identification of a machine-readable data representation may cause the scanning device to self-trigger, capturing image data of the object being scanned for subsequent processing. Alternatively, identification of a machine-readable data representation may cause the scanning device to prompt a user to confirm or otherwise provide input to capture image data. In certain implementations, for example, the user may provide confirmation to the mobile scanning device by pressing a button, touching an icon on a touchscreen, or otherwise providing input. During capture, the flash is deactivated and image data is captured using a camera of the mobile scanning device. The captured image data may then be processed to extract alphanumeric or other data from the image data. Processing the image data may further include storing or transmitting the image data or data extracted from the image data.

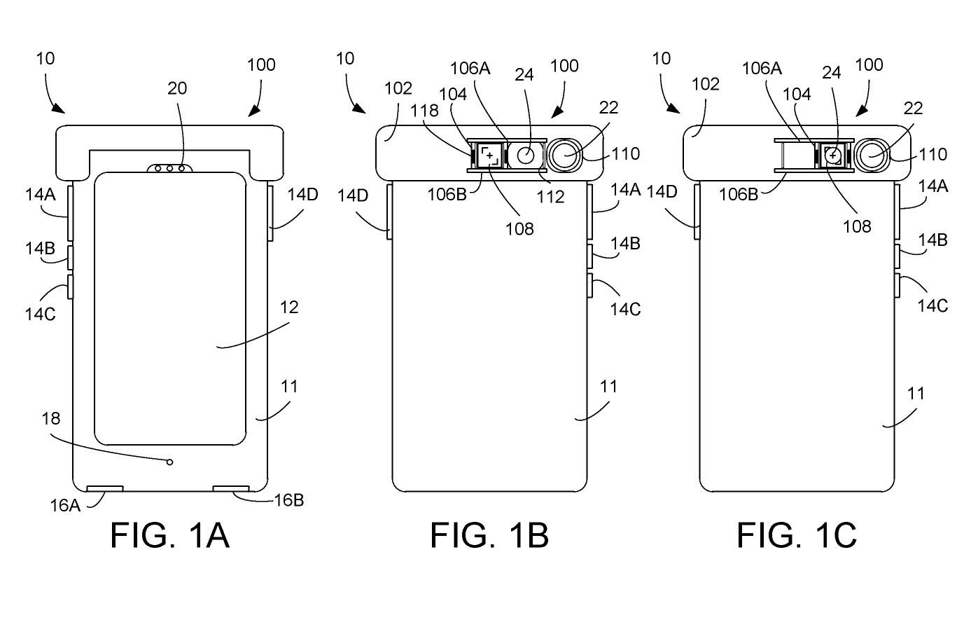

FIGS. 1A-C illustrates a scanning apparatus 10 according to the present disclosure. The scanning apparatus 10 includes a mobile device 11 to which a scanning adapter 100 is coupled. The mobile device 11 may be, among other things, a smart phone or similar mobile computing device. The mobile device 11 includes each of a camera 22 and an associated flash 24 for providing illumination when capturing an image using the camera 22. The mobile device 11 may further include other features and components including, without limitation, one or more of a screen 12 (which may be a touch screen), one or more buttons 14A-D, one or more ports 16A-16B, a microphone 18, and a speaker 20.

The mobile device 11 generally includes a processor and memory including instructions executable by the processor to provide various functions. In implementations of the present disclosure, the memory includes instructions that cause the processor to activate the flash for targeting and to capture and process image data using the camera 22 and the flash 24. Such image data includes barcodes and similar optical, machine-readable representations of data that may be captured using the camera 22. Although the present disclosure primarily uses barcodes as examples of optical, machine-readable representations of data, implementations of the current disclosure are not limited to scanning and processing barcodes or a particular style of barcode. Rather, image data obtained by the mobile device 11 may correspond to any representation of data that may be captured using the camera 22 and processed to obtain the corresponding data. For example, in addition to barcodes (including both one- and two-dimensional (e.g., matrix) barcodes) and QR codes (including both two- and three-dimensional QR codes), such data may include, among other things, plain alphanumeric text and images.

To facilitate improved capture of optical, machine-readable data representations, the scanning apparatus 10 includes a scanning adapter 100 that may be coupled to the mobile device 11. The scanning adapter 100 includes a body 102 defining a camera aperture 110 and a flash aperture 112. As illustrated in FIG. 1B, when the scanning adapter 100 is coupled to the mobile device 11, the camera aperture 110 aligns with the camera 22 and the flash aperture 112 aligns with the flash 24 such that the camera 22 and the flash 24 are unobstructed by the body 102. In certain implementations, the camera aperture 110 and the flash aperture 112 may correspond to different portions of a single aperture defined by the body 102.

In certain implementations, the scanning adapter 100 is coupled to the body 102 by an interference or press fit. In other implementations, the scanning adapter 100 may include bumps, ridges, indentations, and other similar structural features that are positioned and shaped to interact with corresponding features of the mobile device 11. In still other implementations, the scanning adapter 100 may be of a clamshell or similar multi-piece design adapted to close around the mobile device 11.

The scanning adapter 100 further includes a targeting assembly 104 that may be selectively positioned over the flash aperture 112. For example, FIG. 1B illustrates the targeting assembly 104 in a first position in which the targeting assembly 104 is disposed adjacent to the flash 24 and the flash aperture 112 and FIG. 10 the targeting assembly 104 in a second position in which the targeting assembly 104 is disposed over the flash 24 and the flash aperture 112. In the example implementation illustrated in FIGS. 1A-C, the scanning adapter 100 includes a pair of rails 106A-B to which the targeting assembly 104 is mounted and along which the targeting assembly 104 may slide between the first and second positions.

By moving the targeting assembly 104 between the first and second positions, a user of the scanning apparatus 10 may easily transition the scanning apparatus 10 between a non-scanning configuration and a scanning configuration, without decoupling the scanning adapter 100 from the mobile device 11. For example, a user may position the targeting assembly 104 in the second position to perform scanning tasks. If the user subsequently needs to take a picture, conduct a video call, or otherwise use the camera and flash, the user may simply move the targeting assembly 104 to the first position such that the camera and flash are not obstructed.

The targeting assembly 104 generally includes an optical assembly 108 adapted to project a targeting aid in response to activation of the flash 24 when the targeting assembly 104 is disposed in the second position over the flash 24. More specifically, the optical assembly 108 includes one or more optical elements that manipulate light from the flash 24 to project a shape, symbol, design, or other image using the light. The optical assembly 108 is generally configured to have a focal length that is a predetermined distance from the camera 22, such that the projection is in focus at the predetermined distance. The predetermined distance generally corresponds to an optimal or near-optimal distance from the camera 22 for capturing the image data for the particular application and mobile device 11.

In applications in which the mobile device 11 includes an autofocus feature, use of the scanning adapter 100 may improve the overall performance and efficiency of such a feature. For example, many autofocus mechanisms may have a default focus settings that is used as a starting point for performing autofocus. Accordingly, such autofocus mechanisms first adjust to the default focus setting before determining the final focus setting to be used for capturing an image. The scanning adapter 100 may therefore be used to provide a targeting aid that is focused at to or near a distance corresponding to the default focus setting such that the time required to adjust to the final focus setting is significantly reduced.

Moreover, simply having an autofocus function may not result in proper scanning because even if focused, the camera 22 may be positioned too far from the barcode such that the resulting image of the machine-readable data representation is too small (e.g., not enough pixels), thereby making it more difficult or impossible for the mobile device 11 to properly detect the machine-readable data representation. Also, if the camera 22 is placed too close to the target, the captured image may be in focus, but the machine-readable data representation may exceed the field of view of the camera 11 and not be fully contained within the captured image. As a result, the scanning adapter 100 provides significant benefits even when used with a mobile device 11 that may include advanced camera functionality, such as autofocus.

Movement of the targeting assembly 104 between the first and second positions may be facilitated by various structures and features of the targeting assembly 104 and the body 102. For example, as illustrated in FIG. 1B, the targeting assembly 104 may include one or more tabs, such as a tabs 118A,B, indentations, or projections to provide a user with a structure for pushing or pulling the targeting assembly 104 between the first and the second positions. The scanning adapter 100 may further include, among other things, stops, depressions, grooves, and other structural elements that interact with corresponding structural elements of the targeting assembly 104 to prevent or resist movement of the targeting assembly 104 or to positively retain the targeting assembly 104 in one of the first position and the second position. For example, the targeting assembly 104 defines a first groove 152A adapted to receive a corresponding first projection 154A (shown in FIG. 10) extending from the body 102 when the targeting assembly 104 is disposed in the first position and further deigns a second groove 152B adapted to receive a corresponding second projection 154B (shown in FIG. 1B) when the targeting assembly 104 is disposed in the second position.

The implementation of the scanning adapter 100 illustrated in FIGS. 1A-C is in the form of a partial case or cap that is coupled to a top of the mobile device 11 by an interference fit. In other implementations, the scanning adapter 100 may be in the form of a full case that substantially surrounds the mobile device 11. In implementations in which the scanning adapter 100 is a cap or case, the scanning adapter 100 is preferably designed such that the functionality of the mobile device 11 is not limited. For example, the scanning adapter 100 preferably allows actuation of the buttons 14A-D and access to the ports 16A-B even when the scanning adapter 100 is coupled to the mobile device 11. Alternatively, features of the scanning adapter 100 may be directly integrated into the mobile device 11 such that the scanning adapter 100 and the mobile device 11 form a unitary assembly.

Although the scanning adapter 100 is primarily described herein as being usable with a flash 24 of the mobile device 11 that is associated with the camera 22, in other implementations, the scanning adapter 100 is usable with any light source of the mobile device 11. Accordingly, the targeting assembly 104 is more generally movable between a first position in which the optical assembly 108 is not disposed over the light source and a second position in which the optical assembly is disposed over the light source. In light of the foregoing, any specific reference to the flash 24 herein should be understood to also apply to a general light source of the mobile device 11.

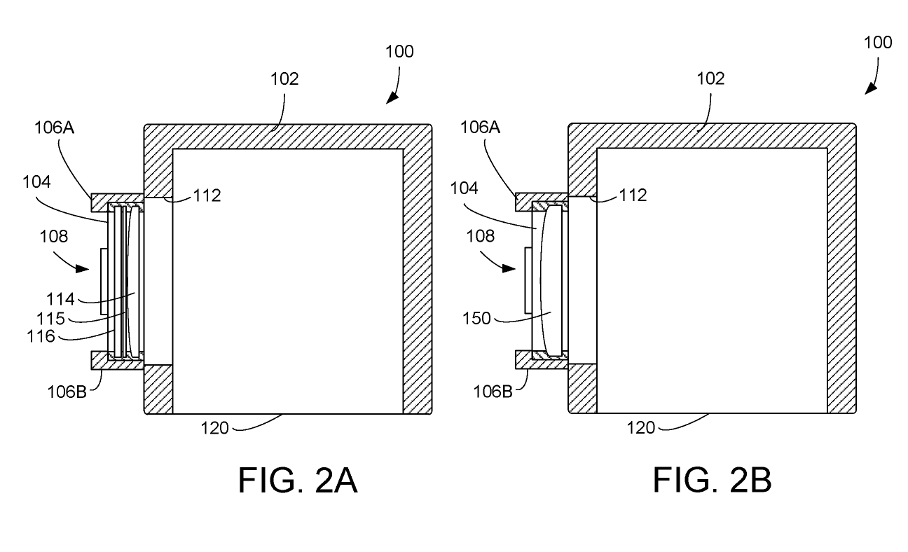

FIGS. 2A-B area cross-sectional side views of implementations of the scanning device 10 and scanning adapter 100 of FIGS. 1A-C taken along section A-A (shown in FIG. 10) and illustrating additional features of the scanning adapter 100. In each of FIGS. 2A-2B the scanning adapter 100 is shown with the targeting assembly 104 in the second position, i.e., placed over the flash aperture 112, as would occur during use of the scanning adapter 100 to provide a targeting aid. For clarity, the mobile device 11 and flash 24 are illustrated in dashed lines.

The body 102 generally defines an opening 120 into which the mobile device 11 may be inserted. The size and shape of the opening 120 and the rest of the body 102 may vary to accommodate various makes and models of mobile devices. Similarly, the size and location of the camera aperture 110 (shown in FIGS. 1B-C) and the flash aperture 112 may similarly be varied based on dimensions of the mobile device for which it is to be used. The body 102 may be formed of various materials including, without limitation, one or more of plastic, rubber, and metal. Because the scanning adapter 100 may function, in part, as a case for the mobile device 11, the body 102 may further be constructed to provide some measure of impact resistance, such as by having a multi-layered construction including a hardened outer shell and an elastomeric lining. In certain implementations, the scanning adapter 100 may also include a coupling mechanism, such as a clip (including, without limitation, a fixed or rotary clip) that may be used to attach the scanning adapter 100 to a piece of clothing or accessory such as a belt. The scanning adapter 100 may also include an anchor or other structure to which a lanyard or similar tether may be attached.

During operation, light from the flash 24 (shown in FIGS. 1A-B) is passed through the optical assembly 108 to produce a visual pattern at a predetermined focal length. As shown in FIG. 2A, the optical assembly 108 may include each of a lens 114, a filter 115, and a mask 116. Generally, the lens 114 causes the light to be focused at the predetermined focal length while the mask 116 causes the light to have a particular shape or design. The filter 115 may be used to, among other things, color or polarize the light as it passes through the optical assembly 108. Alternatively, the filter 115 may be omitted. In certain implementations, the mask 116 may be opaque with a cutout in the shape of the desired light pattern. In other implementations, the mask 116 may be a translucent slide including etching or similar features arranged to direct light into the desired pattern.

The specific order and arrangement of optical elements within the optical assembly 108 may vary between embodiments of scanning adapters in accordance with this disclosure. For example, while the optical assembly 108 depicts the lens 114 being proximal the flash aperture 112 followed by the filter 115 and the mask 116, the order of the lens 114, filter 115, and the mask 116 may be varied. Moreover, any or all of the optical elements of the optical assembly 108 may be combined. For example, FIG. 2B illustrates an alternative configuration of the scanning adapter 100 in which the optical assembly 108 includes a single optical element 150 combining the lens 114, the filter 115, and the mask 116 of FIG. 2A.

Whether or not a filter 115 is used to color the targeting aid may, in certain implementations, be based on the particular algorithms and processing engines implemented by the mobile device 10 to identify and decode machine-readable data representations. For example, certain engines and algorithms may be optimized to ignore certain colors (e.g. red or green) such that the lines of the targeting aid are not confused with elements of the machine-readable data representation. In other cases in which identification and/or decoding is based, at least partially, on color, a white targeting aid may be implemented to avoid inadvertently influencing the algorithms or processing algorithms used to identify and decode.

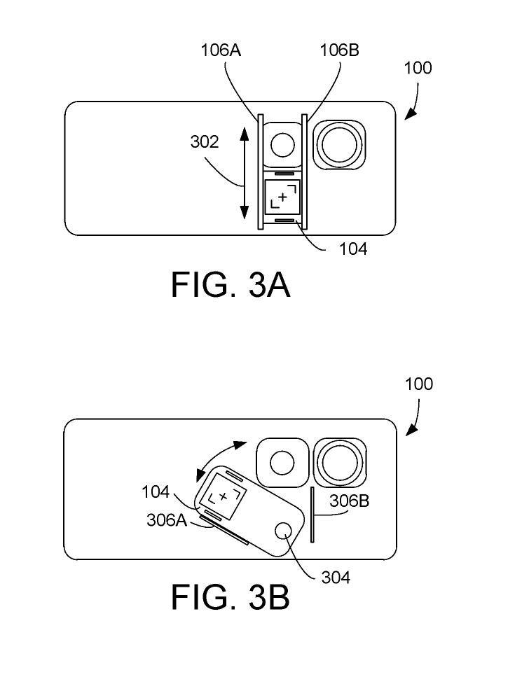

FIGS. 3A-B depict alternative configurations of the scanning adapter 100. In contrast to the configurations of FIGS. 1 and 2, FIG. 3A illustrates a configuration in which the targeting assembly 104 is moved between the first position and the second position is by moving the targeting assembly 104 along the rails 106A, B in a substantially vertical direction 302. FIG. 3B illustrates yet another configuration in which the rails 106A, B are omitted and the targeting assembly 104 is movable between the first and the second positions by rotation of the targeting assembly 104 about a pivot 304. In certain implementations, one or more stops 306A, B may be coupled to or integrated with the body 102 to limit travel of the targeting assembly 104 between the first position and the second position.

In implementations of the present disclosure, the targeting assembly 104 and/or components thereof, such as the optical assembly 108 may be configured to be removable and replaceable with similar components having different projection characteristics such that a user may change targeting or optical assemblies based on the particular scanning task to be performed by the user. For example, a user may perform a first scanning task at an assembly line that requires scanning of a one-dimensional barcode label within approximately three feet. To assist the user in the first scanning task, the user may install a first targeting assembly having a focal length of approximately three feet. The user may then perform a second task involving scanning of two-dimensional barcodes in a warehouse from approximately eight inches away. Accordingly, the user may swap the first targeting assembly with a second targeting assembly designed for the shorter range. As an alternative to or in addition to allowing swapping of targeting assemblies or components thereof, the scanning adapter may include a targeting assembly having multiple optical assemblies that may be selectively disposed in front of the flash of the mobile device. In such implementations, the targeting assembly may be further movable into additional position such that in each position a distinct optical assembly is disposed over the flash.

Changeable targeting and/or optical assemblies may also be of use when the needs of a particular user change over time or differ from a default configuration of the scanning adapter 100. For example, an initial set of targeting and optical assemblies may be provided at a time of initial deployment but the user's specific use case may differ from the characteristics of the default assemblies or may change over time. For example, a shipping clerk may need to scan large barcodes (e.g., six inches across) found on address labels while a nurse may need to scan very small barcodes (e.g., ID bracelets or medicine bottles) such that a targeting and/or optical assembly having a shorter focal length is better suited to the nursing application. Similarly, different scanning applications may require different targeting aids, therefore interchangeable targeting and optical assemblies may be implemented to enable a user to change targeting aids as he or she changes between different scanning tasks.

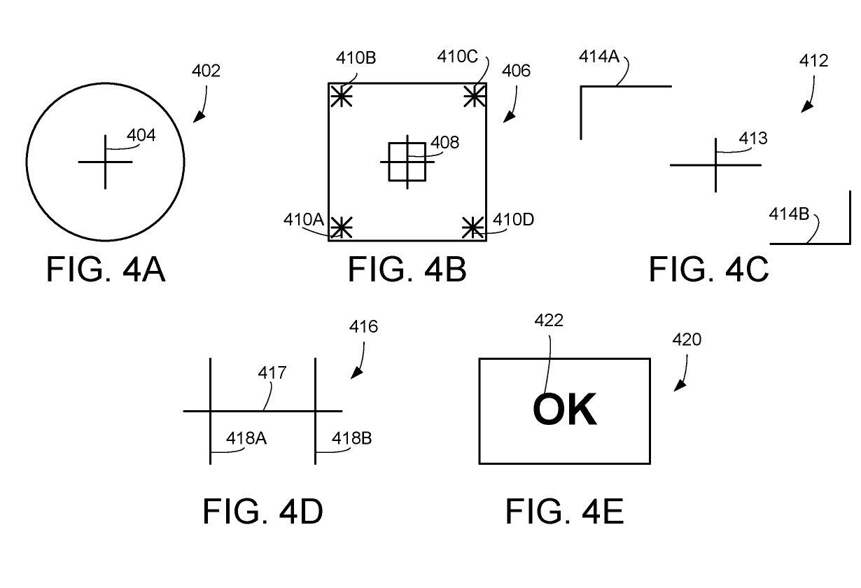

FIGS. 4A-E illustrate example projections that may be generated using scanning adapters in accordance with this disclosure. The example projections of FIGS. 4A-E are not intended to be limiting and merely illustrate certain projections that may be useful in capturing data in conjunction with the scanning adapters. Moreover, although illustrated separately in FIGS. 4A-E, features of any of the foregoing example projections may be used in combination.

FIG. 4A illustrates a circular reticle 402 including a central cross-hair 404 for aligning the circular reticle 402 with a feature of the object being scanned.

FIG. 4B illustrates a square reticle 406 including a cross-hair pattern 408 that may be aligned with a feature of the object being scanned. The square reticle 406 further includes a plurality of focus symbols 410A-D disposed about the square reticle 406 to assist a user in determining whether the user has properly distanced the scanning device from the object being scanned. The focus symbols 410A-D may be, without limitation, shapes, text, pictures, logos, or other visual indicators with sufficient detail such that the detail is only discernable when the scanning device is sufficiently disposed near the optimal scanning distance.

FIG. 4C illustrates a rectangular reticle 412 including a cross-hair 413. The reticle 412 further includes a pair of corners 414A-B. In certain implementations, the reticle 412 may be designed such that the corners 414A-B align with corners of a label or other object containing data to be captured when the scanning device is positioned at a predetermined distance from the object. As a result, the corners facilitate both spacing and orientation of the scanning device relative to the device being scanned. So, if the user positions the scanning device too close or too far from the object being scanned or misaligns the scanning device, the corners 414A-B will generally be out of focus, a different size than the object being scanned, or otherwise misaligned with features of the object being scanned.

FIG. 4D illustrates a linear reticle 416 that may be aligned with a linear data representation, such as a one-dimensional barcode. Similar to the reticle 412 of FIG. 4C, the linear reticle 416 includes a horizontal reticle line 417 and edge guides 418A-B between which a barcode may be disposed during scanning to provide guidance regarding spacing and orientation of the scanning device relative to the object being scanned.

Finally, FIG. 4E illustrates a text-based projection 420 in which text 422 is used instead of a cross-hair. Similar to the focus symbols 408A-D of the square reticle 406 of FIG. 4B, proper distance between the scanning device and the object being scanned may be determined based on the readability of the text 422. In other implementations, the text 422 may be replaced or supplemented with a shape, logo, design, or similar image having features that are discernible when the scanning device is properly positioned relative to the object being scanned.

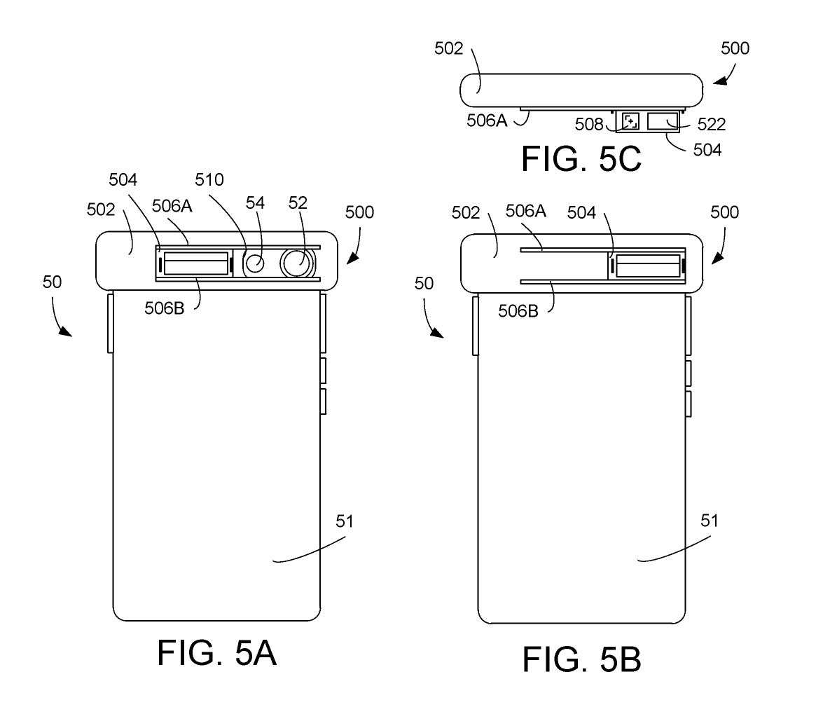

FIGS. 5A-B illustrate a scanning apparatus 50 according to a second embodiment of the present disclosure. The scanning apparatus 50 includes a mobile device 51 to which a scanning adapter 500 is coupled. The mobile device 51 may be, among other things, a smart phone or similar mobile computing device. The mobile device 51 may include each of a camera 52 and an associated flash 54 for providing illumination when capturing an image using the camera 52. Similar to the mobile device 11 of FIGS. 1A-C, the mobile device 51 may further include other features and components including, without limitation, one or more of a screen, one or more buttons, one or more ports, a microphone, and a speaker.

To facilitate improved capture of optical, machine-readable data representations, the scanning apparatus 50 includes a scanning adapter 500 that may be coupled to the mobile device 51. The scanning adapter 500 includes a body 502 defining an aperture 510. As shown in FIG. 5A, when the scanning adapter 500 is coupled to the mobile device 51, the aperture 510 aligns with each of the camera 52 and the flash 54 such that the camera 52 and the flash 54 are unobstructed by the body 502.

The scanning adapter 500 further includes a targeting assembly 504 that may be selectively positioned over the aperture 510. For example, FIG. 5A illustrates the targeting assembly 504 in a first position in which the targeting assembly 504 is disposed adjacent the aperture 510 such that the camera 52 and the flash 54 are not covered by the targeting assembly 504. In FIGS. 5B-C, in contrast, the targeting assembly 504 is in a second position in which the targeting assembly 504 is disposed over the aperture 510, and, as a result, the camera 52 and the flash 54. In the example implementation illustrated in FIGS. 5A-C, the scanning adapter 500 includes a pair of rails 506A-B to which the targeting assembly 504 is mounted and along which the targeting assembly 504 may slide between the first and second positions.

As shown in FIG. 5C, which is a front view of the scanning apparatus 50, the targeting assembly 504 generally includes an optical assembly 508 adapted to project a targeting aid in response to activation of the flash 54 when the targeting assembly 504 is disposed in the second position over the aperture 510. More specifically, the optical assembly 508 includes one or more optical elements that manipulate light from the flash 54 to project a shape, symbol, design, or other image using the light. The optical assembly 508 is generally configured to have a focal length that is a predetermined distance from the optical assembly 508 such that the projection is in focus at the predetermined distance. The predetermined distance generally corresponding to an optimal or near-optimal distance for capturing the image data for the particular application and mobile device 51.

In contrast to the scanning adapter 100 of FIGS. 1A-3B, the scanning adapter 500 is configured to enable use of the scanning apparatus 50 in a substantially horizontal orientation. To facilitate projection of the targeting aid and capture of image data, the targeting assembly 504 may include one or more mirrors for redirecting light from the flash 54 through the optical assembly 508 for purposes of projecting the targeting aid and for redirecting light into the camera 52. As shown in FIG. 5C, the targeting assembly 504 may also include a window 522 or secondary optical assembly aligned with the camera 52 to allow light to reach the camera 52.

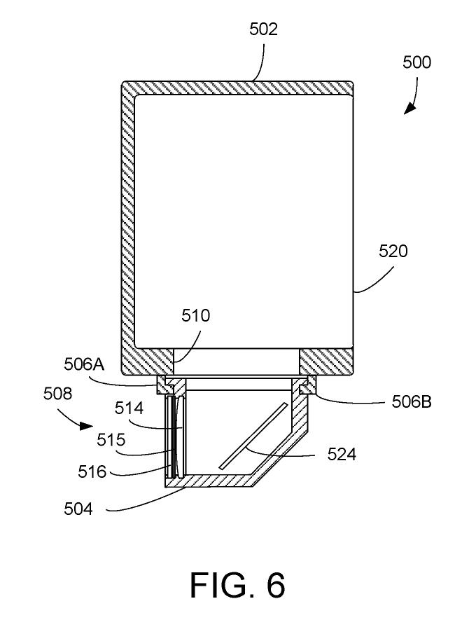

FIG. 6 is a cross-sectional side view of the scanning adapter 500 of FIGS. 5A-C and, in particular, the scanning adapter 500 when the targeting assembly 504 is in in the second position, i.e., placed over the aperture 510.

The body 502 generally defines an opening 520 into which the mobile device 51 may be inserted. During operation, light from the flash 54 (shown in FIG. 5A-B) is passed through the optical assembly 508 to produce a visual pattern at a predetermined focal length. As shown in FIG. 6, the optical assembly 508 may include each of a lens 514, a filter 515, and a mask 516. The specific order and arrangement of optical elements within the optical assembly 508 may vary between embodiments of scanning adapters in accordance with this disclosure. For example, in one alternative configuration of the scanning adapter 500, the optical assembly 508 may include a single optical element combining the lens 514, the filter 515, and the mask 516.

As further illustrated in FIG. 6, the targeting assembly 504 further includes a mirror 524, prism, or other light-redirecting element disposed and supported within the targeting assembly 504. The mirror 524 is oriented within the targeting assembly 504 to redirect light produced by the flash 54 (shown in FIG. 5A) in a substantially perpendicular direction towards the optical assembly 508. The mirror 524 may extend through the targeting assembly 504 to further redirect light entering the targeting assembly through the window 522 (shown in FIG. 5C) into the camera 52 (shown in FIG. 5A). In other implementations, the targeting assembly 504 may include a second mirror dedicated to redirecting light into the camera 52.

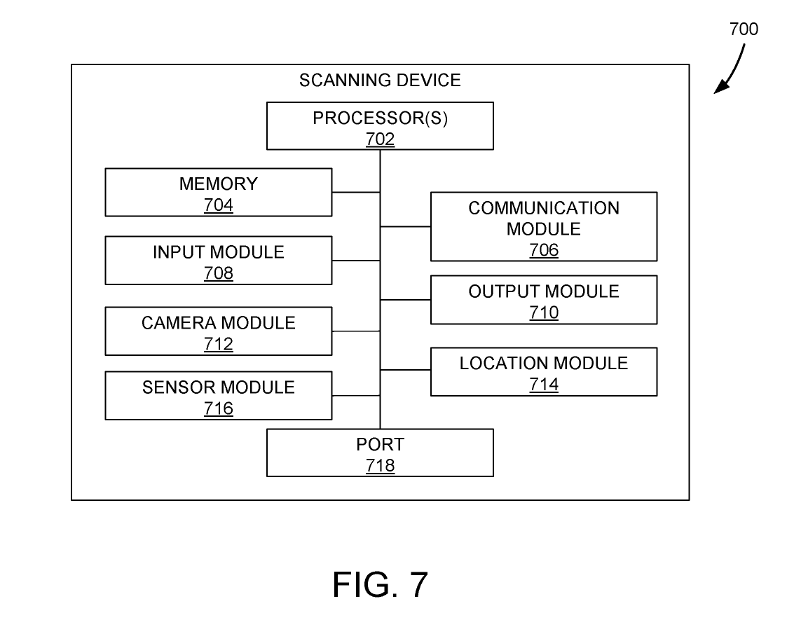

FIG. 7 illustrates a scanning device 700, which may correspond to the scanning devices 10, 50 of FIGS. 1A-C and 5A-C, respectively. The scanning device 700 may be used in conjunction with a scanning adapter in accordance with this disclosure to facilitate capture and processing of image data.

The scanning device 700 is a computing or processing device that includes at least one processor 702 and at least one memory 704 and is preferably a handheld or similar scanning device. The memory 704 generally stores instructions executable by the processor 702 to perform the various functions described herein. The memory 704 may also store data input into or captured using the scanning device 700 including images and data extracted from such images.

The data that may be captured, stored, and processed by the scanning device 700 includes optical machine-readable data including, but not limited to barcodes, QR codes, and similar data representations. To capture such data, the scanning device 700 includes a camera module 712. The camera module 712 may generally include camera hardware and may operate in conjunction with software stored within the camera module 712 or in the memory 704 of the scanning device 700. The camera hardware of the camera module 712 generally includes a flash and may further include one or more of an image sensor, a fixed lens, an adjustable lens, an image processing unit, a memory, and any associate electronic circuitry. The software associated with the camera module 712 generally facilitates capture of an image using the camera hardware and any processing of such an image. In certain implementations, the software may include functionality to convert optical machine-readable data into other data, such as alphanumeric characters.

The scanning device 700 may further include a plurality of modules adapted to perform various functions in conjunction with the processor 702. Such modules may include both software and hardware components. Although more or fewer modules may be included, the scanning device 700 includes, in addition to the camera module 712, each of a communication module 706, an input module 708, an output module 710, a location module 714, and a sensor module 716. The scanning device 700 may further include one or more ports, such as port 718.

The communication module 706 is communicatively coupled to the processor 702 and is generally configured to facilitate communication between the scanning device 700 and other remote computing devices. For example, the communication module 706 may be adapted to wirelessly transmit data to and from the scanning device 700 using one or more of Bluetooth®, Wi-Fi®, IEEE 802.11, ZigBee, near-field communication (NFC), and cellular communication.

The input module 708 allows a user of the scanning device 700 to provide input to the scanning device 700. The input module 708 may include, but is not limited to a keyboard, a touchscreen, an accelerometer, a microphone, or any similar device adapted to receive input from a user of the scanning device 700. The scanning device 700 may further include an output module 710 to provide information to the user. Examples of output devices that may be included in the output module 710 include, but are not limited to a screen, a speaker, and a haptic feedback device. In certain implementations, such as in the case of a touchscreen, the input module 708 and the output module 710 may include shared components.

In certain implementations, the scanning device 700 may include a location module 714 for determining the location of the scanning device 700. The location module 714 may enable the scanning device 700 to determine its location or to provide data to one or more other systems adapted to determine the location of the scanning device 700. In certain implementations, the location module 714 may facilitate communication with the global positioning system (GPS) or similar global location system. The location module 714 may also facilitate communication with local positioning systems for determining the location of a device within a specific area, such as a warehouse or similar facility.

The scanning device 700 may further include a port 718 which may be used to provide one or both of power and data to the scanning device 700. In certain implementations, the port 718 may enable the scanning device 700 to be coupled to a base/docking station or a cable that may in turn be connected to a power source or a computing device to facilitate transmission of data and/or power.

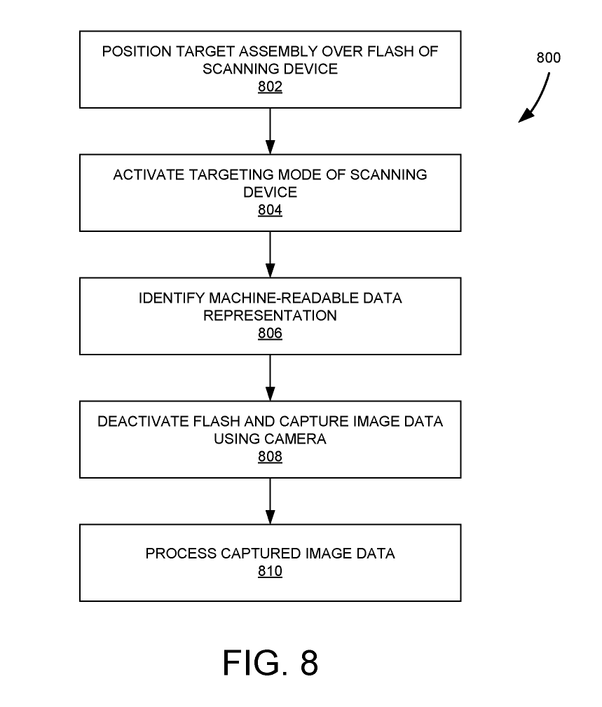

FIG. 8 is a flow chart illustrating a method 800 for capturing and processing image data obtained using a scanning device in accordance with the present disclosure. Reference is made in the following discussion of the method 800 to example elements of the scanning device 10 of FIGS. 1A-2B. Additional reference may be made to elements of the scanning device 50 of FIGS. 5A-6. The method 800 is generally intended to allow a user of the scanning device 10 to project a targeting aid onto an object to be scanned and to capture image data of the object.

During operation, a user of the scanning device 10 may generally load an image capture and processing application or similar software from a memory of the scanning device 10 for execution by a processor of the scanning device 10. In general, the image capture and processing application coordinates the processor, the camera 22 and the flash 24 to project a targeting aid on an object to be scanned and to subsequently capture an image of the object when the scanning device 10 is positioned such that the targeting aid is focused.

At operation 802, the method 800 includes positioning a targeting assembly over a flash of the scanning device. For example, with reference to FIGS. 1A-C, such positioning generally includes sliding the targeting assembly 104 from the first position adjacent the flash aperture 112 (as illustrated in FIG. 1B) to the second position over the flash aperture 112 (as illustrated in FIG. 10).

Next, at operation 804, the scanning device 10 may be placed into a targeting mode in which the flash 24 of the scanning device 10 is activated. Because the targeting assembly 104 is positioned over the flash 24, activation of the flash 24 causes the targeting assembly 104 to project a targeting aid at a predetermined distance from the scanning device 10. The predetermined distance is generally determined by the optical assembly 108 of the targeting assembly 104 and, in particular, the lens 114 of the optical assembly 108.

At operation 806, a machine-readable data representation is identified. In response to being placed into the targeting mode at operation 804, the scanning device 10 may begin to continuously capture and process images from the camera 22. Processing of images from the camera 22 may generally include transmitting or streaming data from the camera 22 to a decoding engine that is capable of identifying and decoding machine-readable data in the images received from the camera 22. In certain implementations, when the decoding engine identifies machine-readable data, a notification or prompt may be presented to the user such that the user may provide a command to capture image data corresponding to the machine-readable data representation using the camera 22. For example, the user may press one of the buttons 14A-D of the scanning device 10, touch a touchscreen of the scanning device 10, or otherwise provide a confirming input to the scanning device. In other implementations, identification of machine-readable data by the decoding engine may automatically trigger a subsequent capture operation.

At operation 808, the flash 24 is deactivated and image data corresponding to the machine-readable data is captured using the camera 22. At operation 810, image data corresponding to the captured image is processed by the scanning device 10 to extract data from the captured image. For example, in the case of capturing a barcode, the scanning device 10 may determine an alphanumeric string represented by the barcode. Processing of the image data may further include storing the image data or data extracted therefrom in a memory of the scanning device 10 and/or transmitting the image data or extracted data to a second computing device.

In certain implementations, the various operations of the method 800 illustrated in FIG. 8 may be aborted or otherwise cancelled by a user. For example, in certain implementations the scanning device 10 may include a scan button or other activation mechanism that, when pressed or otherwise actuated by the user, initiates the method 800. Subsequent release of the activation mechanism or receipt of a cancellation command from the use by the scanning device 10 may then cancel execution of the method 800.

Embodiments of the present disclosure include various operations or steps, which are described in this specification. The steps may be performed by hardware components or may be embodied in machine-executable instructions, which may be used to cause a general-purpose or special-purpose processor programmed with the instructions to perform the steps. Alternatively, the steps may be performed by a combination of hardware, software and/or firmware.

It is believed that the present disclosure and many of its attendant advantages will be understood by the foregoing description, and it will be apparent that various changes may be made in the form, construction, and arrangement of the components without departing from the disclosed subject matter or without sacrificing all of its material advantages. The form described is merely explanatory, and it is the intention of the following claims to encompass and include such changes.

While the present disclosure has been described with reference to various embodiments, it will be understood that these embodiments are illustrative and that the scope of the disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A scanning apparatus comprising:
a computing device comprising a housing, a camera, and a light source, the computing device operable to activate the light source and capture optical data using the camera; and
a scanning adapter coupled to the computing device, the scanning adapter comprising:
a body defining an aperture aligned with the light source; and
a targeting assembly movably coupled to the body between a first position and a second position, the targeting assembly comprising lens, wherein in the first position of the targeting assembly the lens is misaligned with the aperture and in the second position of the targeting assembly the lens is aligned with the aperture and the light source such that a targeting aid is projected at a predetermined distance from the camera in response to activation of the light source.

2. The scanning apparatus of claim 1, wherein the scanning adapter is integrated with the mobile computing device.

3. The scanning apparatus of claim 1, wherein the scanning device further comprises a processor coupled to each of the camera and the light source and a memory communicatively coupled to the processor, the memory storing instructions that, when executed by the processor, cause the processor to:
activate the light source to project the targeting aid;
deactivate the light source in response to receiving a capture command; and
after deactivating the light source, capture image data using the camera.

4. The scanning apparatus of claim 1, wherein, when in the second position, the lens is aligned linearly with each of the light source and the aperture.

5. The scanning apparatus of claim 1, wherein, when in the second position, the lens is aligned at an angle to each of the light source and the aperture, the targeting assembly further comprising a first mirror such that, when in the second position, the first mirror is oriented to reflect light from the light source to the lens.

6. The scanning apparatus of claim 5, wherein, when in the second position, one of the first mirror and a second mirror of the targeting assembly is oriented within the targeting assembly to direct light into the camera.

7. The scanning apparatus of claim 1, further comprising a mask aligned with the lens, wherein the mask defines a shape of at least a portion of the targeting aid.

8. A scanning adapter comprising:
a body defining an aperture; and
a targeting assembly movably coupled to the body between a first position and a second position, the targeting assembly comprising an optical assembly,
wherein, in the first position of the targeting assembly, the optical assembly is misaligned with the aperture and, in the second position of the targeting assembly, the optical assembly is aligned with the aperture such that a targeting aid is projected at a predetermined distance in response to receiving light through the aperture.

9. The scanning adapter of claim 8, wherein, when in the second position, the optical assembly is linearly aligned with the aperture.

10. The scanning adapter of claim 8, wherein, when in the second position, the optical assembly is aligned at an angle to the aperture, the targeting assembly further comprising a first mirror such that, when in the second position, the first mirror is oriented to reflect light entering the aperture to the optical assembly.

11. The scanning adapter of claim 8, wherein the targeting assembly is movably coupled to the body by a pair of rails, the targeting assembly movable between the first position and the second position by sliding the targeting assembly along the rails.

12. The scanning adapter of claim 8, wherein the targeting assembly is movably coupled to the body by a pivot mount, the targeting assembly movable between the first position and the second position by pivoting the targeting assembly about the pivot mount.

13. The scanning adapter of claim 8, wherein the optical assembly comprises:
a lens adapted to focus light entering the aperture at the predetermined distance; and
a mask aligned with the lens and defining a shape of the targeting aid.

14. The scanning adapter of claim 13, wherein the lens and the mask are integrated into a single optical element.

15. The scanning adapter of claim 13, wherein the mask comprises an opaque body defining a cutout shaped to produce the targeting aid.

16. The scanning adapter of claim 13, wherein the mask comprises a translucent body including an etching shaped to produce the targeting aid.

17. The scanning adapter of claim 8, wherein the optical assembly further comprises a filter.

18. The scanning adapter of claim 17, wherein the filter is at least one of a colored filter and a polarizing filter.

19. A method of capturing data using a scanning device, the scanning device comprising a light source, a camera, and a targeting assembly adapted to produce a targeting aid at a predetermined distance from the camera in response to activation of the light source, the method comprising:
positioning the targeting assembly over the light source;
activating the light source;
deactivating the light source in response to receiving a capture command; and
after deactivating the light source, capturing image data using the camera.

20. The method of claim 18 further comprising extracting alphanumeric data from the image data.
